(19) United States
(12) Patent Application Publication
(10) Pub. No.: US 2009/0042982 A1
(43) Pub. Date: Feb. 12, 2009

(54) METHOD FOR TREATING NEUROLOGICAL DISORDERS USING ACETONE DERIVATIVES

(76) Inventors: Serguei S. Likhodi, Toronto (CA); W. Macintyre Burnham, Don Mills (CA)

Correspondence Address: BERESKIN AND PARR, 40 KING STREET WEST, BOX 401, TORONTO, ON, M5H 3Y2, CA

(21) Appl. No.: 12/101,575

(22) Filed: Apr. 11, 2008

Related U.S. Application Data
(63) Continuation of application No. 10/635,447, filed on Aug. 7, 2003, now abandoned.
Application No. 10/635,447 is a continuation-in-part of application No. PCT/CA02/00145, filed on Feb. 7, 2002.
(60) Provisional application No. 60/266,883, filed on Feb. 7, 2001.

(51) Int. Cl. A61K 31/22; A61K 31/121; A61K 31/045; A61P 25/00; A61K 31/11; A61K 31/19

(52) U.S. Cl. 514/546; 514/675; 514/724; 514/557; 514/693

(57) ABSTRACT

The present invention provides a method of treating neurological disorders, such as epilepsy, comprising administering an effect amount of an acetone derivative having the formula R¹—C(X)—R², to an animal in need thereof. Such acetone derivatives show higher anticonvulsant activity, higher potency and improved therapeutic indexes over acetone itself.

### FIELD OF THE INVENTION

The present invention relates to methods of treating neurological disorders including, but not limited to, epilepsy.

### BACKGROUND OF THE INVENTION

Epilepsy is a group of disorders in which spontaneous and recurrent seizures occur. It is one of the most common of neurological conditions, affecting approximately 1% of the population. Epileptic seizures manifest as disruptions of sensation, consciousness, and mental and motor function.

If uncontrolled; seizures can disrupt life to a debilitating degree; status epilepticus can result in permanent neuronal damage or death. In the USA alone, $12.5 billion is spent annually on the direct and indirect costs of epileptic disorders. (For a review, see Annegers 1997.)

The most common therapy for epilepsy is treatment with anticonvulsant drugs. About 15 drugs are currently available. The mechanisms of the anticonvulsants are not completely understood but it appears that these drugs mediate their action either by limiting the spread of epileptic discharge (e.g., carbamazepine) or by elevating the seizure threshold (e.g., ethosuximide). This is achieved through one of three basic mechanisms: 1) binding to voltage-dependent sodium channels and suppressing sodium influx (e.g., carbamazepine and phenyloin); 2) binding to T-type calcium channels and suppressing calcium influx (e.g., ethosuximide); or 3) enhancement of GABAergic activity (e.g., benzodiazepines, vigabatrin). It is quite possible that drugs with activities against many different types of seizures (e.g., valproic acid and many others) have multiple mechanisms of action. (For a discussion of the anticonvulsant drugs, see Burnham 1998.)

The success of therapy with anticonvulsant drugs is high—about 50% of epileptic patients achieve complete seizure control, an additional 25% have significant improvement.

While the anticonvulsants help many epileptics, more than a third of patients continue to suffer from frightening and debilitating seizures, despite the best drug therapy. Seizures which resist anticonvulsant therapy are called “intractable” or “refractory”. There is a clear need for new agents that are effective against intractable seizures.

The introduction of several new anticonvulsants in the past decade raised hopes that intractable seizures might be brought under control. Unfortunately, the seizures that resisted the older drugs have, in general, resisted the new. This is probably because the newer drugs tend work by the same basic mechanisms as the older ones (Löscher 1998).

The following criteria are most important in development of new anticonvulsant drugs: (1) novel mechanism of action; (2) simple pharmacokinetic profile—no interactions with existing drugs; (3) efficacy across the broad spectrum of seizure types; (4) low toxicity and wide therapeutic window.

In the past several years several new drugs have emerged. The mechanism of action of felbamate (1993) is unknown, but may involve inhibition of N-methyl-D-aspartate (NMDA) responses and potentiation of GABA A receptor. Gabapentin is a GABA (gamma-aminobutyric acid) analogue with an unknown mechanism of action, but may involve calcium and sodium channels. The mechanism of action of topirimate (1995) is unknown. It seems have no effect on the level of GABA or glutamate. Tiagabine (1997) is an uptake inhibitor of GABA. The mechanism of action of levetiracetam (1999) is unknown but, after 60 minutes of exposure, it gives rise to increased striata levels of GABA. Remacemide (Phase III) is another sodium channel and a low-affinity NMDA receptor blocker. Owing to its neuroprotective potential, remecemide has been also evaluated in other indications, including Parkinson's and Huntington's diseases (Bialer et al. 2001). Whether the development of these and other new drugs will succeed in combating intractable seizures is unknown at this moment.

To solve the problem of intractable seizures, a novel mechanism of anticonvulsant action should be a principal feature of a new drug. It is known that such mechanisms exist, because intractable seizures can be controlled by certain non-drug therapies. One of these non-drug therapies is the ketogenic diet.

The ketogenic diet is a non-drug therapy, which is surprisingly effective in many different forms of epilepsy (Swink et al. 1997). Although traditionally used in children, it is also effective in adults (Sirven et al. 1999). The diet consists of 3 or 4 parts of fat to 1 part of carbohydrate plus protein (by weight). Due to the low levels of carbohydrate and protein, the ketogenic diet forces the body to utilize fat as its major energy source. This leads to the production of three ketones—acetoacetate, beta-hydroxybutyrate, and acetone—and a state of “ketosis” in the body (Prasad et al. 1996).

The ketogenic diet is an effective treatment for intractable epilepsy, and often the only alternative to surgery (Nordli & DeVivo 1997, Swink et al. 1997). Reports of its successful use over 70 years indicate that at least one third to two thirds of patients with intractable epilepsy benefit substantially from the diet (Keith 1963, Livingston 1972, Kinsman et al. 1992, Nigro et al. 1995, Lefevre & Aronson 2000). The most recent clinical studies have found that about 16% of patients become seizure free on the diet, that about 32% patients have a greater then 90% decrease in seizures, and that 56% of patients have a greater then 50% reduction in seizures (Hemingway et al. 2001). Given that children are not put on the diet unless they have failed to respond to at least two major anticonvulsant drugs, the efficacy of the diet is impressive. In fact, the efficacy of the diet in treating intractable epilepsy significantly exceeds that of the new, recently introduced anticonvulsants (Lefevre & Aronson 2000).

The mechanism of action of the ketogenic diet is not yet understood (although, see below). It is clear, however, that the diet acts by a mechanism different from those of the conventional anticonvulsants. This is indicated by the fact that the diet is effective in cases where all of the known anticonvulsants have failed.

The ketogenic diet has a broad spectrum of anticonvulsant action. It is successful in controlling almost every type of seizure—including seizures that are usually resistant to anticonvulsant drugs, such as complex partial seizures, and the seizures associated with the Lennox-Gastaut syndrome (Swink et al. 1997).

Many clinical studies report a subjective cognitive, psychotropic and behavior improvement in children on the ketogenic diet (Prasad et al. 1996). A recent prospective study has indicated statistically significant beneficial effects of the diet on cognition, behavior and social functioning in children with difficult-to-control seizures (Pulsifer et al. 2001). There are several other reasons to believe that ketogenic diet may have utility as a mood stabilizer (El-Mallakh & Paskifti 2001). These include the observation that some anticonvulsant interventions improve outcome in mood disorders. Such anticonvulsants as carbamazepine, valproate and clonazepam have been found to be effective in a variety of affective disorders including depression and bipolar illness (Ballenger & Post 1980, Lerer et al. 1987, Pope et al. 1991, Bowden et al. 1994, Retzow & Emrich 1998, Dietrich & Emrich 1998). The relationship between depression and epilepsy is two-directional—the patients with major depression also have a higher frequency of epilepsy and vice versa (Kanner & Nieto 1999). Finally, vagal nerve stimulation, a non-drug anticonvulsant procedure, may also be effective in both unipolar and bipolar illness (George et al. 2000).

The ketogenic diet has fewer side effects than most anticonvulsant drugs (Kinsman et al. 1992, Nigro et al. 1995, Mak et al. 1999). The side effects of the ketogenic diet relate mainly to intolerance to the rapid onset of ketosis, hypoglycaemia, refusal to drink fluids, lack of appetite, and nausea. These complications may occur when strict guidelines for the diet administration are not followed and, usually, these problems are easy to correct. A worrying side effect of the ketogenic diet is the rise of serum lipids and cholesterol, which occur in the majority of patients (Rios 2001, Lightstone et al. 2001, Swink et al. 1997).

Although it has few side effects, the ketogenic diet is not easy to maintain. The diet is unpalatable, and some children will not tolerate it. It must also be rigidly followed, since even a slight deviation—such as a single cookie—can provoke a seizure. Children cannot take medications that contain sugar (which is common in many drugs produced for children) (McGhee & Katyal 2001), and must take vitamin supplements to compensate for the diet's nutritional deficiencies. Success with the diet usually depends on patient motivation. Less than 60% of children stay on the diet for 12 months (Hemingway et al. 2001).

Even when successful, children are seldom kept on the diet for more than 3 years, because of its unbalanced nutrition. In adults, the use of the diet is limited by fears of elevated cholesterol (Sirven et al. 1999). What is really needed is a novel anticonvulsant which can reproduce the therapeutic effects of the ketogenic diet, without the rigors of the diet itself.

Identification of the anticonvulsant mechanism of the ketogenic diet is difficult because the diet induces a cascade of metabolic changes. These include ketosis (i.e. elevation of plasma concentrations of beta-hydroxybutyrate, acetoacetate and acetone), changes in electrolytes, pH and water balance, a rise of lipids and fatty acids and other adaptational changes in brain metabolism (Wilder 1921, Lennox 1928, Millichap et al. 1964, Prasad et al. 1996, Schwartzkroin 1999).

Ketosis as a possible casual factor in seizure resistance has not been thoroughly investigated. A few early studies in the 20s and 30s (Wilder 1921, Helmholz & Keith 1930, Keith 1931, 1932a,b) suggested that blood ketones might have anticonvulsant properties. More recent studies on the correlation between the degree of ketosis and seizure resistance, however, have been inconclusive.

Wilder (1921) initially suggested that the anticonvulsant effect of the KD was due to the “sedative” properties of acetoacetate. Experiments of Keith and Helmholz involving the thujone model of epileptic seizures seemed to support Wilder's hypothesis (Helmholz & Keith 1930, Keith 1931, 1932a,b). These experiments suggested that dehydration, acetoacetate and acetone might suppress epileptic attacks (Keith 1931, 1932a,b). The most marked anticonvulsant effects were found with acetoacetic acid and sodium acetoacetate (Keith 1932a). Beta-hydroxybutyrate was not anticonvulsant in this model (Keith 1932b). The Keith's results and conclusions about anticonvulsant activity of acetoacetate are debatable. Acetoacetate does not readily cross the blood brain barrier and, the present inventors have found that it is not effective against pentylenetetrazole and maximal electroshock seizures induced in mice or rats 15-30 min after intraperitoneal injection. At the same time, acetoacetate easily decomposes to form acetone. Therefore, it is possible that the effect of acetoacetate in the Keith's experiments was actually caused by the presence of acetone in the acetoacetate preparation, or was due to acetone formed through the metabolism of acetoacetate.

Huttenlocher (1976) reported that plasma levels of beta-hydroxybutyrate in epileptic children correlated significantly with the anticonvulsant effect of the ketogenic diet. No correlation was demonstrated for acetoacetate, but, nevertheless, the author suggested that either beta-hydroxybutyrate or acetoacetate (or both) might have direct anticonvulsant actions. Recent results in animal models of the ketogenic diet have both supported (Bough & Eagles 1999) and rejected (Bough et al. 1999, Likhodii et al. 2000) a direct relationship between the level of beta-hydroxybutyrate and seizure resistance. Hence, the correlation between beta-hydroxybutyrate and seizure resistance remains inconclusive.

Pretreatment with acetone, however, has protected rats against clonic-tonic convulsions induced by isonicotinic acid and electroshock (Kohli et al. 1967). Acetone given orally to mice seems to reduce the semicarbizide-induced convulsions and mortality (Jenney & Pfeiffer 1958). Two more recent reports from researchers concerned with acetone as an industrial pollutant have suggested that acetone inhibits electrically evoked seizure in 50% of rats exposed for 4 h to air containing acetone vapors (Vodickova et al. 1995, Frantik et al. 1996).

There remains a need for the development of therapeutic agents that can mimic the effect of the ketogenic diet for use in the treatment of epilepsy and other neurological disorders.

### SUMMARY OF THE INVENTION

The present inventors have studied the mechanism of action of the ketogenic diet in a series of animal experiments. Data suggest that the ketogenic diet stops seizures by elevating acetone in the brain. The ketogenic diet elevates blood levels of three ketone bodies, acetoacetate, beta-hydroxybutyrate and acetone. These ketones are also elevated in the brain of rats (see FIG. 1, Likhodii & Burnham, unpublished data) and in the brain of children on the diet (Seymour et al. 1999). The present inventors have found that the diet produces very high concentrations of acetone in blood plasma of human patients receiving ketogenic diet as treatment for epileptic seizures. Acetone concentrations in these patients may reach 2.5-3.0 mmol/L, sometimes 7.0-8.0 mmol/L (see FIG. 2, Likhodii et al., unpublished data). Acetone has proven to be anticonvulsant (Likhodii & Burnham 2002; Likhodii et al. 2003), whereas acetoacetate and beta-hydroxybutyrate have not (Likhodii & Burnham, unpublished data).

The present inventors have further found that acetone suppresses seizures in a number of different animal models, including the maximal electroshock (MES) model (human analog: tonic-clonic seizures), the threshold pentylenetetrazole (PTZ) model (human analog: absence seizures), the amygdala-kindling model (human analog: complex partial seizures with secondary generalization), and the AY-9944 model (human analog, atypical absence, a component of the Lennox-Gastaut syndrome) (see FIG. 3 and Likhodii et al. 2003). The toxicity tests have demonstrated a significant separation between the therapeutic and toxic effects of acetone (Likhodii et al. 2003). Acetone given chronically for 28 days significantly delayed development of kindled seizures in rats and was well tolerated (Likhodii et al., unpublished data). This suggests that acetone may delay or prevent epileptogenesis, i.e. delay or prevent development of epilepsy. One of the most important findings is that sub-toxic doses of acetone are effective against the kindled amygdala focus (see FIG. 3 and Likhodii et al. 2003). The amygdala-kindled focus is a model of complex partial seizures in humans (Albright & Burnham 1980). These are notoriously drug resistant.

The data implicating acetone in actions of the ketogenic diet are in agreement with the reports showing that acetone was significantly elevated in the brain of epileptic children which seizures were controlled by the ketogenic diet (Seymour et al. 1999). The concentrations of acetone in epileptic patients receiving the ketogenic diet treatment are consistent with the degree of the diet's therapeutic effects (Lefevre & Aronson 2000, Hemingway et al. 2001).

The present inventors are therefore the first to confirm the involvement of acetone in the therapeutic effects of the ketogenic diet on epileptic seizures.

It has now been shown that certain acetone derivatives show higher anticonvulsant potency and improved therapeutic indexes over acetone itself. Hence these acetone derivatives are useful in the treatment of epilepsy and other neurological disorders. Specifically, the present inventors have demonstrated that a certain subclass of acetone analogs, wherein the longest continuous carbon chain contains from between 7 and 9 carbon atoms, show improved anticonvulsant potency and/or therapeutic indexes over acetone, butanone, pentanone and their corresponding C3-C5 alcohol derivatives. Surprisingly, it has been found that acetone analogs having a longest continuous carbon chain greater than 9 carbon atoms (C>9, e.g., C10, C11 and C12) do not have anticonvulsant activity.

Accordingly, the present invention provides a method of treating a central nervous system disorder selected from the group consisting of epilepsy, mood disorders, affective disorders and neuropathic pain conditions, comprising administering to an animal in need thereof, an effective amount of a compound of Formula I, or pharmaceutically acceptable solvates thereof:

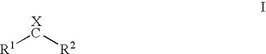

wherein
CX is selected from the group consisting of C═O and CH—OH;
R¹ is selected from the group consisting of branched alkyl, unbranched alkyl, branched alkenyl and unbranched alkenyl;
R² is selected from the group consisting of branched alkyl, unbranched alkyl, branched alkenyl and unbranched alkenyl; and
provided that:
(a) the compound of Formula I contains a longest continuous carbon chain in the range of 7 to 9 carbon atoms; and
(b) “═O” or “—OH” is attached at a position other than a terminal carbon.

The invention also includes the use of an effective amount of compound of Formula I, or pharmaceutically acceptable solvates thereof, to treat a central nervous system disorder. Further, the invention includes a use of an effective amount of a compound of Formula I, or pharmaceutically acceptable solvates thereof, to prepare a medicament to treat a central nervous system disorder.

The compounds of the above structure are useful in replicating therapeutic and anticonvulsant effects of acetone and—by extension—of the ketogenic diet, but have higher potency and/or better therapeutic index (i.e. less side effects) and are more convenient to administer than the ketogenic diet.

Central nervous system disorders that may be treated using the method of the invention include disorders for which a ketogenic diet has shown beneficial effects or disorders which symptoms the ketogenic diet can alleviate. These include, but are not limited to, epilepsy, mood disorders and affective disorders (such as depression, anxiety and unipolar and bipolar illnesses), and neuropathic pain conditions.

Preferably the central nervous system disorder is epilepsy. Accordingly, the present invention provides a method of treating epilepsy comprising administering to an animal in need thereof, an effective amount of a compound of Formula I, or pharmaceutically acceptable solvates thereof. The invention also includes the use of an effective amount of compound of Formula I, or pharmaceutically acceptable solvates thereof, to treat epilepsy. Further, the invention includes a use of an effective amount of a compound of Formula I, or pharmaceutically acceptable solvates thereof, to prepare a medicament to treat epilepsy.

According to another aspect of the present invention, there is provided a pharmaceutical composition comprising a compound of Formula I, or pharmaceutically acceptable solvates thereof, and a pharmaceutically acceptable carrier or diluent.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows portions of proton (1H) NMR spectra from the cerebrospinal fluid (CSF) of a rat fed regular diet (bottom trace), and from a rat fed the ketogenic diet (top trace). The spectra suggest significant elevation of brain acetone due to the ketogenic diet.

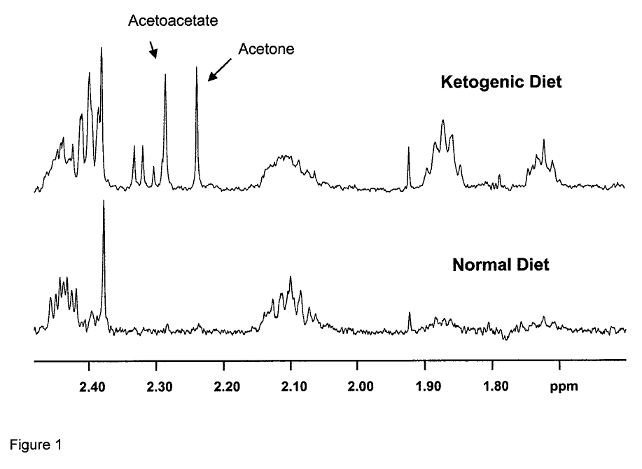

FIG. 2 shows portions of proton (1H) NMR spectra from control blood plasma (human healthy volunteer consuming regular diet, bottom trace), and from the plasma of an epileptic patient receiving the ketogenic diet (top trace). The diet produces high acetone concentrations in humans. Acetone concentrations in plasma in human patients may reach 2.5-3.0 mmol/L, sometimes 7.0-8.0 mmol/L.

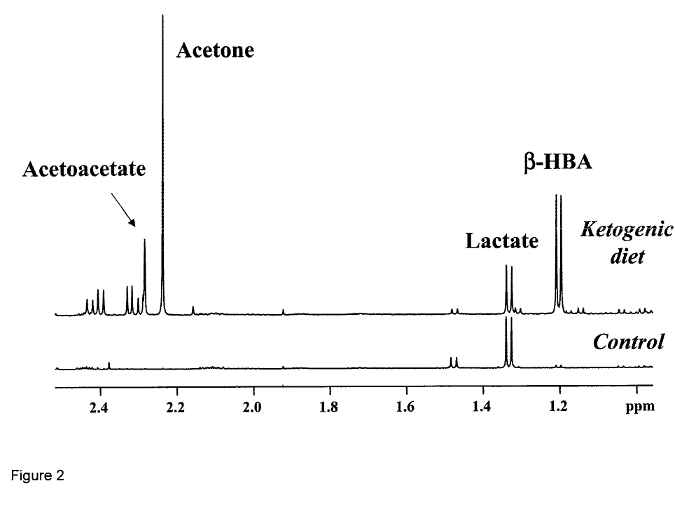

FIG. 3 includes graphs showing the dose-response curves for acetone's anticonvulsant effects in (a) maximal electroshock (MES), (b) pentylenetetrazole (PTZ), (c) amygdala kindling and (d) AY-9944 models of seizure. Panels (a), (b) and (c) show percent of animals protected from seizures as a function of logarithm of the acetone dose. The AY-9944-induced seizures were quantified by measurement of the onset-to-offset durations of the slow spike-and-wave discharge (SWD) combined over the 20-min intervals. In the AY-9944 model, average ‘% SWD decrease’ was the response parameter. The ‘% SWD decrease’ was defined for each subject as 100% (SWD(baseline)−SWD(acetone))/SWD(baseline), where SWD(baseline) and SWD(acetone) are the averaged baseline and the post-injection SWD durations, respectively, quantified over the 20-min intervals. Panel (d) shows the ‘% SWD decrease’ as a function of logarithm of the acetone dose.

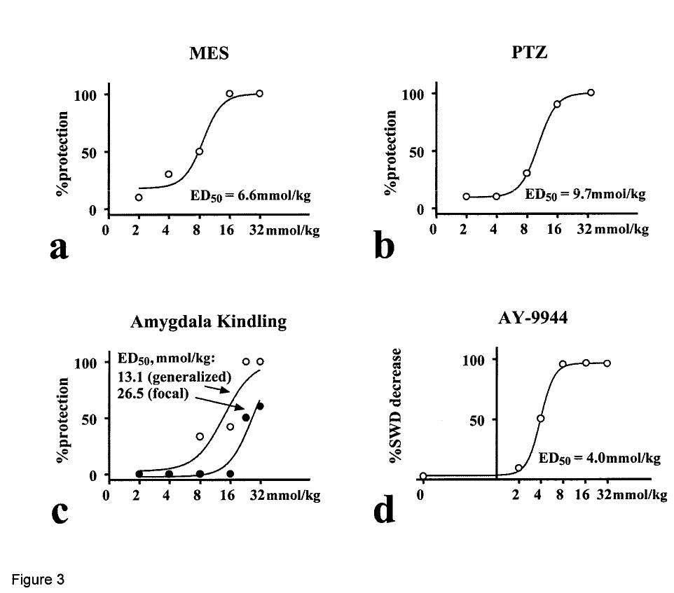

FIG. 4 shows representative dose-response curves for selected compounds from Table 1 obtained using maximal electroshock (MES) seizure test (filled circles, left curves) and rotorod toxicity test (open squares, right curves). % Response shown in each panel represents percent of animals protected from seizures (left curves) or percent of animals that exhibited toxicity and failed the rotorod test (right curves).

FIG. 5 shows potency (ED₅₀) for suppression of experimental seizures—(a), and therapeutic index (TI)— (b) as functions of a number of carbons in carbon chains of 2-ketones and 2-alcohols (see also Table 1). The potency increases with chain elongation from 2-butanone to 2-hexanone, reaches “local” maximum at 2-hexanone, then somewhat decreases for 2-octanone (a, left panel). The therapeutic index has “local” minimums for compounds with even number of carbons (b, right panel). There appears therefore to be a preference for compounds of Formula I having an odd number of carbon atoms (i.e. 7 or 9) in the longest continuous carbon chain.

### DETAILED DESCRIPTION OF THE INVENTION

### I. Method of the Invention

The present inventors have shown that the ketogenic diet elicits its therapeutic effects by elevating acetone in the brain. In particular, they have shown that acetone injected intraperitoneally, raises seizure threshold in animal models of epileptic seizures.

The inventors have not only found that acetone is anticonvulsant, they have further found that acetone—like the ketogenic diet—has a broad spectrum of action. It suppresses seizures in a number of different animal models, including the maximal electroshock (MES) model (human analog: tonic-clonic seizures), the threshold pentylenetetrazole (PTZ) model (human analog: absence seizures), the amygdala-kindling model (human analog: complex partial seizures with secondary generalization), and the AY-9944 model (human analog, atypical absence, a component of the Lennox-Gastaut syndrome).

For the first time, the present inventors have shown that modified acetone-like compounds are useful as anticonvulsants. These modified acetone compounds show higher anticonvulsant potency and/or improved therapeutic indexes over acetone itself. Specifically, the present inventors have demonstrated that a certain subclass of acetone analogs, wherein the longest continuous carbon chain contains from between 7 and 9 carbon atoms, show improved anticonvulsant potency and/or therapeutic indexes over acetone, butanone, pentanone and their corresponding C₃-C₅ alcohol derivatives.

Surprisingly, it has been found that acetone analogs having a longest continuous carbon chain greater than 9 carbon atoms (C>9, e.g. C10, C11 and C12) do not have anticonvulsant activity.

Accordingly, the present invention provides a method of treating a central nervous system disorder selected from the group consisting of epilepsy, mood disorders, affective disorders and neuropathic pain conditions, comprising administering to an animal in need thereof, an effective amount of a compound of Formula I, or pharmaceutically acceptable solvates thereof:

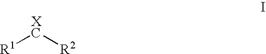

wherein
CX is selected from the group consisting of C═O and CH—OH;
R¹ is selected from the group consisting of branched alkyl, unbranched alkyl, branched alkenyl and unbranched alkenyl;
R² is selected from the group consisting of branched alkyl, unbranched alkyl, branched alkenyl and unbranched alkenyl; and
provided that:
(a) the compound of Formula I contains a longest continuous carbon chain in the range of 7 to 9 carbon atoms; and
(b) “═O” or “—OH” is attached at a position other than a terminal carbon.

The invention also includes the use of an effective amount of compound of Formula I, or pharmaceutically acceptable solvates thereof, to treat a central nervous system disorder. Further, the invention includes a use of an effective amount of a compound of Formula I, or pharmaceutically acceptable solvates thereof, to prepare a medicament to treat a central nervous system disorder.

Central nervous system disorders that may be treated using the method of the invention are those for which a ketogenic diet has shown beneficial effects or disorders which symptoms the ketogenic diet can alleviate. These include, but are not limited to, epilepsy, mood disorders and affective disorders (such as depression, anxiety and unipolar and bipolar illnesses), and neuropathic pain conditions.

The compounds of Formula I have been shown to act as effective anticonvulsants. Accordingly, the present invention provides a method of treating convulsions comprising administering an effective amount of a compound of Formula I, or pharmaceutically acceptable solvates thereof, to an animal in need thereof. The invention also includes the use of an effective amount of compound of Formula I, or pharmaceutically acceptable solvates thereof, as an anticonvulsant. Further, the invention includes a use of an effective amount of a compound of Formula I, or pharmaceutically acceptable solvates thereof, to prepare a medicament to treat convulsions.

Preferably the central nervous system disorder is epilepsy. Accordingly, the present invention provides a method of treating epilepsy comprising administering to an animal in need thereof, an effective amount of a compound of Formula I, or pharmaceutically acceptable solvates thereof. The invention also includes the use of an effective amount of compound of Formula I, or pharmaceutically acceptable solvates thereof, to treat epilepsy. Further, the invention includes a use of an effective amount of a compound of Formula I, or pharmaceutically acceptable solvates thereof, to prepare a medicament to treat epilepsy.

The term an “effective amount” or a “sufficient amount” of an agent as used herein is that amount sufficient to effect beneficial or desired results, including clinical results, and, as such, an “effective amount” depends upon the context in which it is being applied. For example, in the context of administering an agent that is an anticonvulsant, an effective amount of an agent is, for example, an amount sufficient to achieve such a reduction in convulsions as compared to the response obtained without administration of the agent.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

To “inhibit” or “suppress” or “reduce” a function or activity, such as convulsions, is to reduce the function or activity when compared to otherwise same conditions except for a condition or parameter of interest, or alternatively, as compared to another conditions.

The term “animal” as used herein includes all members of the animal kingdom including human. The animal is preferably a human.

The term “pharmaceutically acceptable” as used herein means to be compatible with the treatment of animals, in particular humans.

The term “solvate” as used herein means a compound of Formula I wherein molecules of a suitable solvent are incorporated in the crystal lattice. A suitable solvent is physiologically tolerable at the dosage administered. Examples of suitable solvents are ethanol, water, oil and the like. When water is the solvent, the molecule is referred to as a “hydrate”.

The term “alkyl” as used herein refers to a saturated carbon chain [i.e.—(CH₂)ₙCH₃)]. The term “alkenyl” as used herein refers to carbon chains containing one or more double bonds (or units of unsaturation). When R¹ and/or R² is branched or unbranched alkenyl, the carbon chain may contain any number of double bonds, including compounds of Formula I that are fully unsaturated as well as those that contain only 1 double bond. It is preferred that, when R¹ and/or R² is branched or unbranched alkenyl in a compound of Formula I, that the compound of Formula I contain 1 or 2 double bonds, more preferably 1 double bond. It is a preferred embodiment of the present invention that both R¹ and R² are branched or unbranched alkyl.

The compounds of Formula I extend to cover ketones and alcohol derivatives of acetone containing a carbon chain ranging from 7 to 9 carbons in length (including the carbon to which the ═O or —OH is attached). The ═O or —OH may be attached to the carbon chain at any position accept at a terminal carbon. In a preferred embodiment, the ═O or —OH is attached at the “2-position” (i.e. the second carbon from the terminus) of R¹ or R². The carbon chain may be branched and the invention extends to all such branched compounds of Formula I provided that the longest continuous carbon chain contains between 7 and 9 carbon atoms. In a preferred embodiment of the invention, the longest continuous carbon chain contains 7 or 9 carbon atoms. In preferred embodiments of the present invention, CX is C═O.

Some of the compounds of Formula I may have at least one asymmetric center. Where the compounds of Formula I have one asymmetric center, they may exist as enantiomers. Where the compounds of Formula I possess two or more asymmetric centers, they may additionally exist as diastereomers. It is to be understood that the use of all such isomers and mixtures thereof in any proportion are encompassed within the scope of the present invention.

In further embodiments of the present invention, the compounds of Formula I for use in the methods of the present invention are selected from the group consisting of:
4-heptanone;
2-heptanone;
2-octanone;
5-nonanone;
4-nonanone;
3-nonanone;
2-nonanone;
2-nonanol;
2-octanol; and
2-heptanol.

Preferably, the compounds of Formula I for use in the methods of the present invention are selected from the group consisting of:
5-nonanone;
4-nonanone;
2-nonanone;
2-nonanol; and
2-heptanol.

Compounds may be examined for their efficacy as anticonvulsants using a number of different animal models, including the maximal electroshock (MES) model (human analog: tonic-clonic seizures) as described in Krall et al. (1978) and in Example 1 herein, the threshold pentylenetetrazole (PTZ) model (human analog: absence seizures) as described in Krall et al. (1978), the amygdala-kindling model (human analog: complex partial seizures with secondary generalization) as described in Albright & Burnham (1980), and the AY-9944 model (human analog, atypical absence, a component of the Lennox-Gastaut syndrome) as described in Cortez et al. (2001). The compounds may also be tested for their toxicity using standard assays, such as the standard rotorod assay as described in Dunham & Miya (1957) and Wlaz & Löscher (1998) and in Example 1 herein. Based on the results of the anticonvulsant assay (typically expressed in units of ED₅₀) and the toxicity assay (typically expressed in units of TD₅₀) a therapeutic index may be calculated for each compound which is the ratio of TD₅₀/ED₅₀. The larger the therapeutic index, the more desirable the compound for use in the methods of the invention.

The compounds of Formula I are preferably formulated into pharmaceutical compositions for administration to human subjects in a biologically compatible form suitable for administration in vivo. Accordingly, in another aspect, the present invention provides a pharmaceutical composition comprising a compound of Formula I, or pharmaceutically acceptable solvates thereof, in admixture with a suitable diluent or carrier.

The compositions containing the compounds of Formula I, or pharmaceutically acceptable solvates thereof, can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

In accordance with the methods of the invention, the described compounds of Formula I, or pharmaceutically acceptable solvates thereof, may be administered to a patient in a variety of forms depending on the selected route of administration, as will be understood by those skilled in the art. The compounds or compositions of the invention may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A compound Formula I, or pharmaceutically acceptable solvates thereof, may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound Formula I may be incorporated with excipient and used in the form of ingestible tablets, buccal tablets, troches, capsules, elixirs, suspensions, syrups, wafers, and the like.

A compound Formula I, or pharmaceutically acceptable solvates thereof, may also be administered parenterally or intraperitoneally. Solutions of a compound Formula I, or pharmaceutically acceptable solvates thereof, can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (1990-18th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The dosage of the compounds of Formula I, or pharmaceutically acceptable solvates thereof, and/or compositions of the invention can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One skilled in the art can determine the appropriate dosage based on the above factors. The compounds of the invention may be administered initially in a suitable dosage that may be adjusted as required, depending on the clinical response.

The compounds of the invention can be used alone or in combination with other agents that have anticonvulsant activity or in combination with other types of treatment for epilepsy or other neurological disorders. Ill. Methods of Preparing Compounds of Formula I Compounds of Formula I are either commercially available or may be prepared using standard procedures known to a person skilled in the art. For example, compounds of Formula I, wherein CX is CH—OH) may be prepared from the corresponding ketones using standard reducing agents such as hydride reducing agents. Correspondingly, compounds of Formula I wherein CX is C═O are available from their corresponding alcohols using standard oxidation conditions or from a corresponding olefin by oxidation (see for example, Monflier et al. 1995, Alper et al. 1985). Alternate syntheses of compounds of Formula I, may be found in, for example, the following references: Kamimura, Y. et al. (2000); Takikawa, H. et al. (1997); Cherkaoui, H. et al. (2001); Macho, V. et al. (1998); Jewett, D. K. et al. (1996); Ebert & Klein (1991); Markevich, V. S. et al. (1985); Arase, A. (1984); Brown & Wetherill (1993).

In some cases the chemistries outlined above may have to be modified, for instance by use of protective groups, to prevent side reactions due to reactive groups, such as reactive groups attached as substituents. This may be achieved by means of conventional protecting groups, for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 1991.

The formation of solvates of the compounds of Formula I will vary depending on the compound and the solvate. In general, solvates are formed by dissolving the compound in the appropriate solvent and isolating the solvate by cooling or using an antisolvent. The solvate is typically dried or azeotroped under ambient conditions.

Prodrugs of the compounds of Formula I may be conventional esters formed with an available hydroxy group. For example, when in a compound of Formula I, CX is CH—OH, the OH group may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters.

The following non-limiting examples are illustrative of the present invention:

### EXAMPLES

### Example 1

### Anticonvulsant Activity and Therapeutic Index Objective

The objective of these experiments was to measure the anticonvulsant activity of compounds structurally related to acetone. The measurements established the structure-activity relationship, potency and toxicity and the therapeutic index.

Methods.

Male CF-1 mice, weighing 28-30 g, were used as subjects (Charles River Canada, La Prairie, Quebec, Canada). Three to five subjects per drug dose were used to prescreen each compound for anticonvulsant activity. Prescreening involved injecting subjects with the drug at doses of 3, 6 mmol/kg and assessing activity and toxicity 30 min later. Promising compounds progressed to dose-response studies, which employed at least 50 subjects, 10 subjects per dose.

Compounds were dissolved in oil and injected intraperitoneally. Preliminary experiments with sham injections confirmed that oil, used as a vehicle in these experiments, did not have anticonvulsant activity.

Toxicity of the injected drugs was assessed using the standard rotorod test (Dunham & Miya 1957, Wlaz & Löscher 1998). The test was administered about 25 minutes after the injection. In brief, the diameter of a rotating rod was about 5 cm and the number of revolutions per minute was set at 6 rpm. Mice were placed on the rod so that it was rotated toward the animal. Animals that were not able to maintain their equilibrium on the rod for 1 min were again put on the rod a further two times. Only mice that were unable to stay on the rod three sequential 1-min trials were considered to exhibit a neurological deficit.

Thirty minutes after drug injection, the MES seizure test was administered. We used the procedure of Krall et al. (1978). In brief, a seizure was induced using the electrical current applied via corneal electrodes. The current was set to 50 mA with a 60 Hz sine wave pulse configuration and train duration of 0.2 sec. Seizure protection was defined in this model as a failure to extend the hind limbs to an angle greater than 90 degrees during the tonic period of the convulsion.

Data analysis was performed using GraphPad Prizm software package version 3.02 for Windows (GraphPad Software Inc, San Diego, Calif., USA). Both dose-response and toxicity data were fitted using non-linear sigmoidal dose-response model with variable slope: Y=Bottom+(Top-Bottom)/(1+10̂((LogED₅₀−X)*HillSlope)), where Bottom, Top, HillSlope and ED₅₀ were the fitting parameters, X was the logarithm of drug concentration, and Y was the response parameter. ED₅₀ and TD₅₀, which are the concentrations that give a response in 50% subjects in seizure and toxicity tests, were determined as best-fit values. Therapeutic index (TI) was calculated as a ratio of TD₅₀/ED₅₀. Results and Discussion.

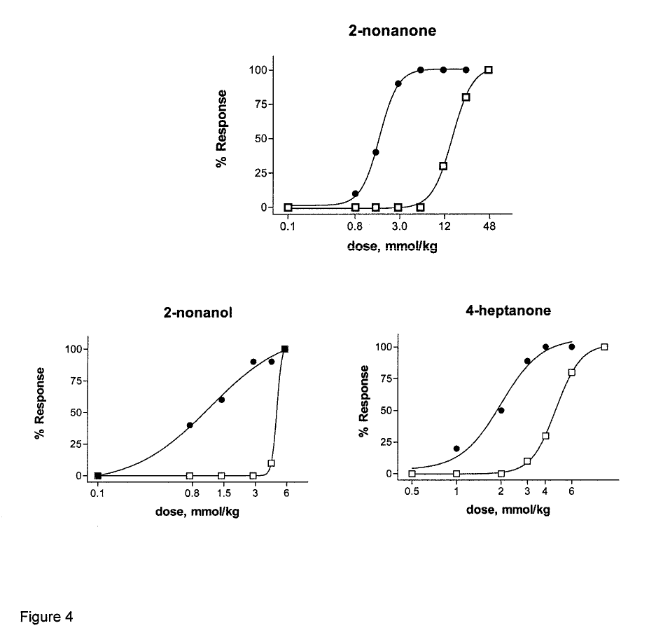

FIG. 4 displays the results of some of the dose-response experiments and corresponding non-linear sigmoidal fits for each data set. Table 1 summarizes the MES dose-response and rotorod toxicity data. Structurally, the tested compounds differ from acetone due to the extension of the length/number of carbons either on one (Series I) or both (Series II) of the aliphatic chains, or by shifting (Series III) or replacing the keto group (Series IV). Table 1 also provides data on acetone derivatives that were not active as anticonvulsants (Table 1, Series V).

The ED₅₀ and TD₅₀ values in the Table 1 are given as mmol/kg of body weight. The procedures were first validated using the standard anticonvulsant, valproate (ED₅₀=1.4 mmol/kg, TD₅₀=3.8 mmol/kg, TI=2.7). Five series of experiments then explored the effects of structural variations on activity.

The data suggest a specific binding site for acetone, since small changes in structure cause large changes in anticonvulsant activity (see Table 1). The following data indicate the involvement of a protein receptor: 1) the cooperativity effect (steep slope) for Series II, and 2) the sudden increase in potency for 2-nonanone (Series III). Series IV suggests that ═O and —OH may play a role in binding to the receptor. Some alcohols are already known to interact with selective neural proteins, including ion channels, kinases and transporters (Harris 1999). The absence of anticonvulsant activity in such acetone analogues as diacetone alcohol, 1,4-pentanediol, methoxyacetone and ethyl acetoacetate (Table 1, Series V) suggests the role of a hydrophobic site in binding to a receptor; incorporation of electronegative O or OH into hydrophobic carbon chain abolished anticonvulsant activity. Compounds with the ═O group attached at terminus of the carbon chain (C1 position) were also inactive as anticonvulsants—these examples include nonanal and hexanal (Table 1, Series V). Hydrocarbons, i.e. carbon chains without ═O or —OH attached, were also inactive as anticonvulsant—these examples include nonane (see Table 1, Series V).

As Table 1 indicates, most of the compounds tested are as potent or more potent than acetone, which has an ED₅₀ of 16 mmol/kg using the methods described herein. Many also have a better therapeutic index than acetone, which has an index of 2.2 using the methods described herein. In fact, many also have a better therapeutic index than the standard anticonvulsant, valproate, which has a therapeutic index of 2.7 using the methods described herein. One of the newly discovered compounds (2-nonanone) has an index of 9.4, which is 3.5 times better than valproate.

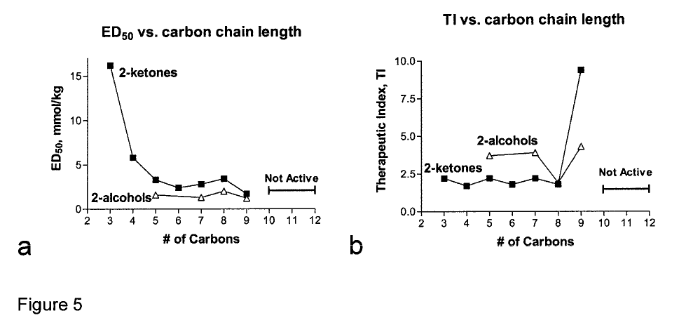

The data illustrates that compounds of Formula I having a longest continuous carbon chain of 10 carbon atoms or longer are not active as anticonvulsants in the assays performed here. This ‘cutoff’ of anticonvulsant effect occurring at carbon chains longer than 9 carbons (C>9) in both ketones and their alcohol analogues is reported here for the first time (see FIG. 5, a).

While not wishing to be limited by theory, the ‘cutoff’ phenomena (loss or plateau in potency of a series of alcohols as carbon chain is increased beyond a certain point) can be attributed to actions of ketones and alcohols on both lipid membranes and protein receptors (see Peoples et al. 1996). The ‘cutoff’ effects could be explained, as proposed by Franks & Lieb (1985), by the existence of hydrophobic binding pockets of limited sizes located in receptor proteins and acting as binding sites.

The ‘cutoff’ phenomena have been reported for various effects of alcohols. These include effects of alcohol intoxication for which the intoxicating potency of aliphatic n-alcohols reaches a plateau and decreases as the number of carbons increases from six to eight (McCreery & Hunt 1978; Lyon et al. 1981).

Much attention has been recently focused on neurotransmitter-gated ion channels as potential sites of alcohol action. Alifimoff et al. (1993), for example, reported effects of secondary alcohols from 2-butanol to 2-octanol on nicotinic acetylcholine receptors. They confirmed that 2-alcanols, similar to 1-alcanols, exerted two actions on the acetylcholine ion channel. The short-chain alkanols augmented agonist affinity without inhibiting the channel, whereas the longer chain alcanols inhibited the channel. The IC₅₀ for 2-butanol was about 60-70 mmol/L, the IC₅₀ for 2-octanol was about 90 umol/L (about 1000× difference). The IC₅₀ for 2-butanol and large difference in IC₅₀'s between 2-butanol and 2-octanol suggest that effects in nicotinic acetylcholine receptors differ from the anticonvulsant effects reported herein. First, the IC₅₀ for 2-butanol's effect on nicotinic receptors was much higher then any of the concentrations in our experiments. Second, alcanols have been reported to exhibit a ‘cutoff’ effect in their actions on acetylcholine receptors at C>12 (McKenzie et al. 1995), not at C>9 as in the present case.

The ‘cutoff’ effects on GABA A, NMDA, AMPA (alpha-amino-3-hydroxy-5-methyl-4-isoxazole-propionic acid), ATP (adenosine triphosphate) and other receptor-ion channels have also been reported (for review see Peoples et al. 1996). Interestingly, the ‘cutoff’ points for different receptors differ but none of them appear to occur at C>9. For example, a ‘cutoff’ for GABA A occurs at 12 or 13 carbon atoms, while a ‘cutoff’ for NMDA receptor occurs at C>8 carbon atoms (see Peoples et al. 1996). The mechanism of the ‘cutoff’ effect in the present experiments therefore is unclear.

The present data indicate that potency of both ketones and alcohols at suppressing seizures increases with elongation of the carbon chain (FIG. 5, a). The potency in the Series II (see Table 1 and FIG. 5, a) increases with chain elongation from 2-butanone to 2-hexanone, reaches “local” maximum at 2-hexanone, then somewhat decreases for 2-octanone. Compounds with 7 carbons (C7) in the longest continuous chain appear to be significantly more potent than compounds with 5 (C5) or with less number of carbon atoms (see Table 2). The potency sharply increases for 2-nonanone (FIG. 5, a). Interestingly, the therapeutic index in this Series, reaches ‘local’ minimums for compounds with even number of carbons (see FIG. 5, b and Table 1, Series II). The TI's for 2-butanone, 2-hexanone, 2-octanone are 1.7-1.8; these are worse than the acetone's TI). The TI's for compounds with odd number of carbons, in contrast, are equal to or significantly better than that for acetone. The TI in this Series II reaches maximum for 2-nonanone. There appears therefore to be a preference for compounds of Formula I having an odd number of carbon atoms (i.e. 7 or 9) in the longest continuous carbon chain. Conclusions.

The data on structure-activity relationships suggest that the proposed anticonvulsants work by the same mechanism as their parent compound—acetone. The mechanism of their action, therefore, replicates the unique effect of the ketogenic diet capable of antagonizing epileptic seizures, which do not respond to current drug therapies. The proposed anticonvulsants, which have higher potency and better therapeutic index than acetone, may significantly improve the control of intractable seizures.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Summary of the structure-activity, dose-response experiments with
acetone analogs.

| | | | | |
|---|---|---|---|---|
| | | ED₅₀ | TD₅₀ | |
| Name | Structure | (slope) | (slope) | TI |

Series I-Elongating both side chains

| | | | | |
|---|---|---|---|---|
| Acetone | 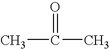 | 16.2 (60.3) | 35.2(16.1) | 2.2 |
| 3-Pentanone | 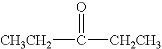 | 3.3 (16.9) | 7.6(14.7) | 2.3 |
| 4-Heptanone | 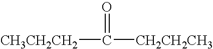 | 2.0 (3.2) | 4.7 (5.2) | 2.4 |
| 5-Nonanone | 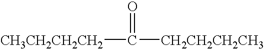 | 3.6 (2.2) | 13.2(11.0) | 3.6 |
| 6-Undecanone | 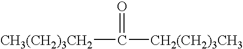 | NotActive | NotActive | NotActive |

Series II-Elongating a side chain

| | | | | |
|---|---|---|---|---|
| 2-Butanone | 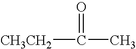 | 5.8(114.6) | 10.0(12.6) | 1.7 |
| 2-Pentanone | 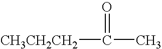 | 3.3 (11.5) | 7.2 (7.7) | 2.2 |
| 2-Hexanone | 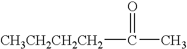 | 2.4 (6.5) | 4.4 (4.9) | 1.8 |
| 2-Heptanone | 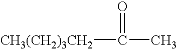 | 2.8 (1.7) | 6.0 (2.0) | 2.2 |
| 2-Octanone | 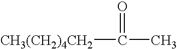 | 3.4 (10.5) | 6.0(57.5) | 1.8 |
| 2-Nonanone | 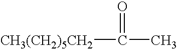 | 1.7 (3.5) | 15.9 (3.3) | 9.4 |
| 2-Decanone | 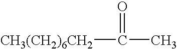 | NotActive | NotActive | NotActive |
| 2-Undecanone | 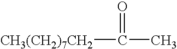 | NotActive | NotActive | NotActive |

Series III-Shifting C═O position

| | | | | |
|---|---|---|---|---|
| 5-Nonanone | 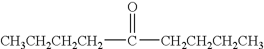 | 3.6 (2.2) | 13.2(11.0) | 3.6 |
| 4-Nonanone | 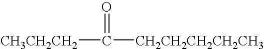 | 5.0 (4.5) | 16.3 (4.9) | 3.3 |
| 3-Nonanone | 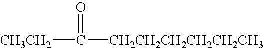 | 9.6 (5.4) | 20.9 (4.5) | 2.2 |
| 2-Nonanone | 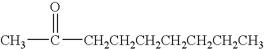 | 1.7 (3.5) | 15.9 (3.3) | 9.4 |

Series IV-Replacing ═O with —OH

| | | | | |
|---|---|---|---|---|
| 2-Decanol | 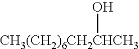 | NotActive | NotActive | NotActive |
| 2-Nonanol | 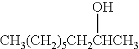 | 1.2 (1.2) | 5.0(23.3) | 4.3 |
| 2-Octanol | 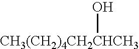 | 2.0 (61.7) | 3.9(20.6) | 1.9 |
| 2-Heptanol | 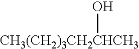 | 1.3 (4.1) | 5.1(10.7) | 3.9 |
| 2-Pentanol | 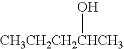 | 1.6 (63.9) | 5.7(37.1) | 3.7 |

| | |
|---|---|
| Name | Structure |

Series V-Inactive analogues

| | |
|---|---|
| 2-OctenoicAcid | 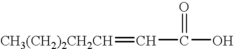 |
| Diacetone | 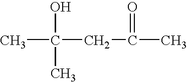 |
| Butylacetate | 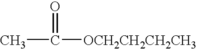 |
| Methoxyacetone | 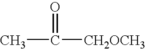 |
| 1,4-Pentanediol | 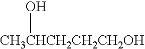 |
| Ethyl acetoacetate | 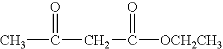 |
| Hexanal | 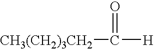 |
| Nonanal | 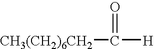 |
| Nonane | CH₃CH₂(CH₂)₅CH₂CH₃ |
| Methyl heptanoate | 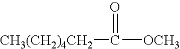 |

Notes for Table 1.
The ED₅₀ and TD₅₀ values in the Table 1 are given as mmol/kg of body weight. Techniques were first validated using the standard anticonvulsant, valproate (ED₅₀ = 1.4 mmol/kg, TD₅₀ = 3.8 mmol/kg, TI = 2.7).

TABLE 2
Comparison of potencies of the C5- vs. C7-long-chain ketones and
alcohols.

| | | | Two- | Signif- |
|---|---|---|---|---|
| 2-pentanol | 2-heptanol | | tailed P | icant? |
| LogED₅₀ = 0.2331 | LogED₅₀ = 0.1197 | t = 4.371 | 0.0047 | Yes |
| SE = 3.910e−005 | SE = 0.02593 | with 6 df | | |
| | | | Two- | Signif- |
| 3-pentanone | 4-heptanone | | tailed P | icant? |
| LogED₅₀ = 0.5124 | LogED₅₀ = 0.2981 | t = 3.898 | 0.0176 | Yes |
| SE = 0.009794 | SE = 0.05407 | with 4 df | | |
| | | | Two- | Signif- |
| 2-pentanone | 2-heptanone | | tailed P | icant? |
| LogED₅₀ = 0.5176 | LogED₅₀ = 0.4447 | t = 3.897 | 0.0080 | Yes |
| SE = 0.009402 | SE = 0.01616 | with 6 df | | |

Note: LogED₅₀'s were compared using t-test with the help of GraphPad Prism version 3.00 for Windows, GraphPad Software, San Diego California USA, www.graphpad.com. Abbreviations: SE = standard error, df- degrees of freedom.

### FULL CITATIONS FOR REFERENCES REFERRED TO IN THE SPECIFICATION

Albright P S, Burnham W M. (1980) Development of a new pharmacological seizure model: effects of anticonvulsants on cortical- and amygdala-kindled seizures in the rat. Epilepsia, 21:681-9.
Alifimoff J K, Bugge B, Forman S A, Miller K W. (1993) Stereoselectivity of channel inhibition by secondary alkanol enantiomers at nicotinic acetylcholine receptors. Anesthesiology, 79:122-8.
Alper, H, Januszkiewicz, K, Smith D J H. (1985) Palladium chloride and polyethylene glycol promoted oxidation of terminal and internal olefins. Tetrahedron Lett. 26:2263-64.
Annegers J F. Epidemiology of epilepsy. In: Wyllie E, ed. The treatment of epilepsy: principles and practice. 2nd ed. Baltimore: Williams & Wilkins, 1997:165-72.
Arase, A, Hoshi M. Masuda, Y. (1984) A novel synthesis of internal alkenyldialkylborane by the reaction of 1-halo-1-alkenyldialkylborane with Grignard reagent. Bull. Chem. Soc. Jpn. 57:209-213.
Bialer M, Johannessen S I, Kupferberg H J, Levy R H, Loiseau P, Perucca E. (2001) Progress report on new antiepileptic drugs: a summary of the Fifth Eilat Conference (EILAT V). Epilepsy Res. 43:11-58.
Ballenger J C, Post R M. (1980) Carbamazepine in manic-depressive illness: a new treatment. Am J Psychiatry, 37:782-90.
Bough K J, Eagles D A (1999) A ketogenic diet increases the resistance to pentylenetetrazole-induced seizures in the rat. Epilepsia 40:138-43.
Bough K J, Chen R S, Eagles D A (1999) Path analysis shows that increasing ketogenic ratio, but not beta-hydroxybutyrate, elevates seizure threshold in the rat. Dev Neurosci 21:400-6.
Bowden C L, Brugger A M, Swann A C et al. (1994) For the Depakote Mania Study Group. Efficacy of divalproex vs. lithium and placebo in the treatment of mania. J Am Med Assoc, 271:918-24.
Brown, H. C. and Wetherill, R. B. (1993) Highly specific synthesis of [E]- and [Z]-3-alkyl substituted allylboronates via one-carbon homologation of stereospecific 1-alken-1-ylboronates. Tetrahedron Lett. 34:7845-8
Burnham W M. Antiseizure drugs (Anticonvulsants). In: Principles of Medical Pharmacology. Kalant H and Roschlau W H E, eds., Oxford Press, pp. 250-261, 1998.
Cherkaoui, H., Soufiaoui, M, Gree, R. (2001) From allylic alcohols to saturated carbonyls using Fe(CO)₅ as a catalyst: scope and limitation studies using preparation of two perfume compounds. Tetrahedron, 57:2379-2383.
Cortez M A, McKerlie C, Snead O C 3rd. (2001) A model of atypical absence seizures: EEG, pharmacology, and developmental characterization. Neurology, 56:341-9.
Dietrich D E, Emrich H M. (1998) The use of anticonvulsants to augment antidepressant medication. J Clin Psychiatry, 59:51-8.
Dunham N W and Miya T S. (1957) A note on a simple apparatus for detecting neurological deficit in mice and rats. J Am Pharm Assoc, 46:208.
Ebert, G W, Klein, W R. (1991) Direct formation and subsequent substitution of remote ketone-functionalized organocopper reagents. J. Org. Chem. 56:4744-7.
El-Mallakh R S and Paskitti M E. (2001) The ketogenic diet may have mood-stabilizing properties. Medical Hypotheses, 57:724-6.
Franks N P, Lieb W R. (1985) Mapping of general anaesthetic target sites provides a molecular basis for cutoff effects. Nature, 316:349-51.
Frantik E, Vodickova L, Hornychova M, Nosek M. (1996) Pattern of inhalation exposure: blood levels and acute subnarcotic effects of toluene and acetone in rats. Cent Eur J Public Health, 4:226-32.
George M S, Sackeim H A, Rush A J et al. (2000) Vagus nerve stimulation: a new tool for brain research and therapy. Biol Psychiatry, 47:287-95.
Harris R A. (1999) Ethanol actions on multiple ion channels: which are important? Alcohol Clin Exp Res, 23:1563-70.
Helmholz H F and Keith H M. (1930) Eight years' experience with the ketogenic diet in the treatment of epilepsy. JAMA, 95:707-9.
Hemingway C, Freeman J M, Pillas D J, Pyzik P L. (2001) The ketogenic diet: a 3- to 6-year follow-up of 150 children enrolled prospectively. Pediatrics, 108:898-905.
Huttenlocher P R. (1976) Ketonemia and seizures: Metabolic and anticonvulsant effects of two ketogenic diets in childhood epilepsy. Pediatr Res, 10:536-40.
Jenney E H, Pfeiffer C C. (1958) The convulsant effect of hydrazides and the antidotal effect of anticonvulsants and metabolites. J Pharmacol Exp Ther, 122:110-23.
Jewett, D K, Brigham, D L, Bjostad, L B. (1996) Hesperophylax occidentalis (Trichoptera: Limnephilidae): electroantennogram structure-activity study of sex pheromone component 6-methylnonan-3-one. J. Chem. Ecol. 22.123-37.
Kamimura, Y, Sato, S, Takahashi, R, Sodesawa, T, Fukui, M. (2000) Vapor-phase synthesis of symmetric ketone from alcohol over CeO₂—Fe²O₃ catalysts. Chem. Lett. 3:232-233.
Kanner A M, Nieto J C. (1999) Depressive disorders in epilepsy. Neurology, 53:S26-32.
Keith H M. (1931) The effect of various factors on experimentally produced convulsions. Am J Dis Child, 41:532-43.
Keith H M. (1932a) Effects of various substances on convulsions due to thujone. Am J Dis Child, 44:682-3.
Keith H M. (1932b) Further studies of the control of experimentally produced convulsions. J Pharm Exper Therap, 44:449-55.
Keith H M. Convulsive disorders in children—with reference to treatment with ketogenic diet. Boston, Little, Brown, 1963.
Kinsman S L, Vining E P, Quaskey S A, Mellits D, Freeman J M. (1992) Efficacy of the ketogenic diet for intractable seizure disorders: review of 58 cases. Epilepsia, 33:1132-6.
Kohli R P, Kishor K, Dua P R, Saxena R C. (1967) Anticonvulsant activity of some carbonyl containing compounds. Indian J Med Res, 55:1221-5.
Krall R L, Penry J K, White B G, Kupferberg H J, Swinyard E A. (1978) Antiepileptic drug development: II. Anticonvulsant drug screening. Epilepsia, 19:409-28.
Lefevre F, Aronson N. (2000) Ketogenic diet for the treatment of refractory epilepsy in children: A systematic review of efficacy. Pediatrics, 105:E46.
Lennox W G (1928) Ketogenic diet in the treatment of epilepsy. N Engl J Med, 199:74-5.
Lerer B, Moore N, Meyendorff E, Cho S R, Gershon S. (1987) Carbamazepine versus lithium in mania: a double-blind study. J Clin Psychiatry, 48:89-93.
Lightstone L, Shinnar S, Callahan C M, O'Dell C, Moshe S L, Ballaban-Gil K R. (2001) Reasons for failure of the ketogenic diet. J Neurosci Nurs, 33:292-5.
Likhodii S S, Musa K, Mendonca A, Dell C, Burnham W M, Cunnane S C (2000) Dietary fat, ketosis, and seizure resistance in rats on the ketogenic diet. Epilepsia, 41:1400-10.
Likhodii S S, Burnham W M. (2002) Ketogenic diet: does acetone stop seizures? Med Sci Monit, 8:HY19-24.
Likhodii S S, Serbanescu I, Cortez M A, Murphy P, Snead O C 3rd, Burnham W M. (2003) Anticonvulsant properties of acetone, a brain ketone elevated by the ketogenic diet. Ann Neurol, 54:219-26.
Livingston S. Dietary Treatment for Epilepsy. In: Livingston S. Comprehensive management of epilepsy in infancy, childhood and adolescence. Springfield, Ill.: Charles C. Thomas; 1972:378-405.
Löscher W. (1998) New visions in the pharmacology of anticonvulsion. Eur J Pharmacol, 342:1-13.
Lyon R C, McComb J A, Schreurs J, Goldstein D B. (1981) A relationship between alcohol intoxication and the disordering of brain membranes by a series of short-chain alcohols. J Pharmacol Exp Ther, 218:669-75.
Macho, V., Jurecek, L, Komora, L, Jurecekova, E. (1998) Preparation of higher aliphatic and/or cycloaliphatic ketones. SK 279092.
Mak S C, Chi C S, Wan C J. (1999) Clinical experience of ketogenic diet on children with refractory epilepsy. Acta Paediatr Taiwan, 40:97-100.
Markevich, V S, Stepanova, G A, Vinogradov, M G. (1985) Conjugated addition of acetaldehyde and acetone to olefins. 25:64-7.
McCreery M J, Hunt W A. (1978) Physico-chemical correlates of alcohol intoxication. Neuropharmacology, 17:451-61.
McGhee B, Katyal N. (2001) Avoid unnecessary drug-related carbohydrates for patients consuming the ketogenic diet. J Am Diet Assoc, 101:87-101.
McKenzie D, Franks N P, Lieb W R. (1995) Actions of general anaesthetics on a neuronal nicotinic acetylcholine receptor in isolated identified neurones of Lymnaea stagnalis. Br J Pharmacol, 115:275-82.
Millichap J G, Jones J D, Rudis B P (1964) Mechanism of anticonvulsant action of ketogenic diet. Am J Dis Child, 107:593-604.
Monflier, E, Tilloy, S, Fremy, G, Barbaux, Y, Mortreux, A. (1995) A very useful and efficient Wacker oxidation of higher alpha-olefins in the presence of per(2,6-di-O-methyl)-beta-cyclodextrin. 36:387-8.
Nigro M A, Ventimiglia J, Selcen D, Beierwaltes P. (1995) Seizure frequency, behavioral and performance effects of the ketogenic diet. Ann Neurology, 38:549-50.
Nordli D R Jr, De Vivo D C. (1997) The ketogenic diet revisited: back to the future.Epilepsia, 38:743-9.
Peoples R W, Li C, Weight F F. (1996) Lipid vs protein theories of alcohol action in the nervous system. Annu Rev Pharmacol Toxicol, 36:185-201.
Pope H G Jr, McElroy S L, Keck P E Jr, Hudson J I. (1991) Valproate in the treatment of acute mania: a placebo-controlled study. Arch Gen Psychiatry, 48:62-8.
Prasad A N, Stafstrom C F, Holmes G L. (1996) Alternative epilepsy therapies: the ketogenic diet, immunoglobulins, and steroids. Epilepsia, 37:S81-95.
Pulsifer M B, Gordon J M, Brandt J, Vining E P, Freeman J M. (2001) Effects of ketogenic diet on development and behavior: preliminary report of a prospective study. Dev Med Child Neurol, 43:301-6.
Remington's Pharmaceutical Sciences, Mack Publishing Company, Easton, Pa., USA 1985.
Retzow A, Emrich H M. (1998) Therapy of bipolar affective illnesses with valproate. A review of the literature. Psychiatr Prax, 25:163-71.
Rios V G. (2001) Complications of treatment of epilepsy by a ketogenic diet. Rev Neurol, 33:909-15.
Vodickova L, Frantik E, Vodickova A (1995) Neutrotropic effects and blood levels of solvents at combined exposures: binary mixtures of toluene, o-xylene and acetone in rats and mice. Cent Eur J Public Health, 3:57-64.
Seymour K J, Bluml S, Sutherling J, Sutherling W, Ross B D. (1999) Identification of cerebral acetone by 1H-MRS in patients with epilepsy controlled by ketogenic diet. MAGMA, 8:33-42.
Sirven J, Whedon B, Caplan D, Liporace J, Glosser D, O'Dwyer J, Sperling M R. (1999) The ketogenic diet for intractable epilepsy in adults: preliminary results. Epilepsia, 40:1721-6.
Schwartzkroin P A. (1999) Mechanisms underlying the anti-epileptic efficacy of the ketogenic diet. Epilepsy Res, 37:171-80.
Swink T D, Vining E P, Freeman J M. (1997) The ketogenic diet: 1997. Adv Pediatr, 44:297-329.
Takikawa, H, Tamagawa, H, Mori, K. (1997) Pheromone synthesis. Part-189: Synthesis of the enantiomers of 6-methyl-3-nonanone, the femail-produced sex pheromone of Caddisfly, Hesperophylax occidentalis. J. Indian Che, Soc. 74:855-57.
Wilder R M. (1921) The effects of ketonemia on the course of epilepsy. Mayo Clin Proc, 2:307-8.
Wlaz P and Löscher W. Evaluation of associated behavioral and cognitive deficits in anticonvulsant drug testing. In: Neuropharmacology methods in epilepsy research, Eds. Peterson S L and Albertson T E. CRC Press, New York, 1998, pp. 171-192.

1. A method of treating a central nervous system disorder selected from the group consisting of epilepsy, mood disorders, affective disorders and neuropathic pain conditions, comprising administering to an animal in need thereof, an effective amount of a compound of Formula I, or pharmaceutically acceptable solvates thereof: 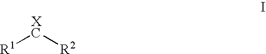
wherein
CX is selected from the group consisting of C═O and CH—OH;
R¹ is selected from the group consisting of branched alkyl, unbranched alkyl, branched alkenyl and unbranched alkenyl;
R² is selected from the group consisting of branched alkyl, unbranched alkyl, branched alkenyl and unbranched alkenyl; and
provided that:
(a) the compound of Formula I contains a longest continuous carbon chain in the range of 7 to 9 carbon atoms; and
(b) “═O” or “—OH” is attached at a position other than a terminal carbon.

2. The method according to claim 1, wherein the longest continuous carbon chain in a compound of Formula I contains 7 or 9 carbon atoms.

3. The method according to claim 2, wherein the longest continuous carbon chain in a compound of Formula I contains 9 carbon atoms.

4. The method according to claim 1, wherein R¹ and R² are independently selected from the group consisting of branched alkyl and unbranched alkyl.

5. The method according to claim 4, wherein R¹ and R² are both unbranched alkyl.

6. The method according to claim 1, wherein the compound of Formula I contains a maximum of 1 double bond.

7. The method according to claim 1, wherein the “═O” or “—OH” is attached at the C2 position of the longest continuous carbon chain.

8. The method according to claim 1, wherein CX is C═O.

9. The method according to claim 1, wherein the compound of Formula I is selected from the group consisting of:
4-heptanone;
2-heptanone;
2-octanone;
5-nonanone;
4-nonanone;
3-nonanone;
2-nonanone;
2-nonanol;
2-octanol; and
2-heptanol.

10. The method according to claim 1, wherein the compound of Formula I is selected from the group consisting of:
5-nonanone;
4-nonanone;
2-nonanone;
2-nonanol; and
2-heptanol.

11. The method according to claim 1, wherein the neurological disorder is selected from the group consisting of epilepsy, depression, anxiety, unipolar illness and bipolar illnesses.

12. The method according to claim 1, wherein the neurological disorder is epilepsy.

13. (canceled)

14. The method according to claim 1, where in CX is CH—OH.

15. The method according to claim 14, wherein the compound of Formula I is selected from the group consisting of 2-nonanol, 2-octanol and 2-heptanol.

16. The method according to claim 14, wherein the compound of Formula I is 2-nonanol or 2-heptanol.
